(19) United States
(12) Patent Application Publication
(10) Pub. No.: US 2020/0171001 A1
(43) Pub. Date: Jun. 4, 2020

(54) COMBINATION THERAPY WITH 2,3-DIHYDRO-ISOINDOLE-1-ONE COMPOUNDS AND METHODS FOR TREATING PATIENTS WITH VARIOUS MUTATIONS

(71) Applicant: Aptose Biosciences Inc., Mississauga (CA)

(72) Inventor: William G. RICE, Del Mar, CA (US)

(21) Appl. No.: 16/700,426

(22) Filed: Dec. 2, 2019

Related U.S. Application Data
(60) Provisional application No. 62/773,686, filed on Nov. 30, 2018.

(51) Int. Cl. A61K 31/4178; A61K 31/496; A61K 31/5377; A61K 31/495; A61K 9/20; A61P 35/02

(52) U.S. Cl. CPC A61K 31/4178; A61K 31/496; A61P 35/02; A61K 31/495; A61K 9/20; A61K 31/5377

(57) ABSTRACT

The present disclosure comprises a method for administering 2,3-dihydro-isoindole-1-one compound or a pharmaceutically acceptable salt, ester, solvate and/or prodrug thereof, alone or in combination with an anticancer agent, for the treatment of hematological cancers such as acute myeloid leukemia (AML). The present disclosure further relates to reducing or inhibiting mutated IDH1 activity in a subject.

### CROSS REFERENCE TO RELATED APPLICATIONS

This application claims the priority to U.S. Provisional Application No. 62/773,686, filed Nov. 30, 2018, the disclosure of which is hereby incorporated by reference in its entirety for all purposes.

### FIELD OF THE INVENTION

The present invention relates to a 2,3-dihydro-isoindole-1-one compound, or pharmaceutically acceptable salts, esters, prodrugs, hydrates, solvates and isomers thereof for the treatment of cancers, such as hematologic cancers, where the patients exhibit IDH1 mutations.

### BACKGROUND OF THE INVENTION

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2, 4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM 005896.2 and NP_005887.2 respectively. The nucleotide and amino acid sequences for IDH1 are also described in, e.g., Nekrutenko et al., Mol. Biol. Evol. 15: 1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMB L/GenB ank/DDB J databases; and Sjoeblom et al, Science 314:268-274(2006).

Non-mutant, e.g., wild type, IDH1 catalyzes the oxidative decarboxylation of isocitrate to α-ketoglutarate (α-KG) thereby reducing NAD⁺(NADP⁺) to NADH (NADPH), e.g., in the forward reaction:

Isocitrate+NAD⁺(NADP⁺)→α-KG+CO₂+NADH(NADPH)+H⁺.

It has been discovered that mutations of IDH1 present in certain cancer cells result in a new ability of the enzyme to catalyze the NAPH-dependent reduction of α-ketoglutarate to R(−)-2-hydroxyglutarate (2HG). The production of 2HG is believed to contribute to the formation and progression of cancer (Dang, L et al, Nature 2009, 462:739-44).

The inhibition of mutant IDH1 is therefore a potential therapeutic treatment for cancer. Accordingly, there is an ongoing need for inhibitors of IDH1 mutants. This invention meets that need.

### SUMMARY OF THE INVENTION

The present disclosure relates to Compound 7, pharmaceutically acceptable salts, esters, prodrugs, hydrates, solvates and isomers thereof.

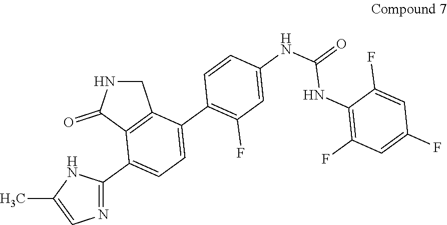 [SMILES: Cc1cnc(-c2ccc(-c3ccc(NC(=O)Nc4c(F)cc(F)cc4F)cc3F)c3c2C(=O)NC3)[nH]1]

In some embodiments, the present disclosure provides a method of inhibiting or reducing mutated IDH1 activity or expression in a subject comprising administering Compound 7 or a pharmaceutically acceptable salt thereof. In some embodiments, the mutated IDH1 comprises at least one point mutation. For example, the at least one point mutation is on one or more residues selected from the group consisting of G97X, R100X, R132X, H133X, and A134X, wherein X means the possibility of any amino acid. In some embodiments, the G97X mutation is G97D and/or the H133X mutation is H133Q, and/or the A134X mutation is A134D. In some embodiments, the R132X mutation is R132H or R132C. In some embodiments, the R132X mutation is R132H. In some embodiments, the at least one point mutation is two or more point mutations present on the same allele. In some embodiments, the at least one point mutation is two or more point mutations present on different alleles. In some embodiments, the subject is a mammal (e.g. a human).

In some embodiments, the methods of the present disclosure further includes inhibiting or reducing wild type or mutant Fms-related tyrosine kinase 3 (FLT3) activity or expression in a subject in need thereof. In some embodiments, the FLT3 is mutated. For example, in some embodiments, the mutated FLT3 comprises at least one point mutation (e.g. the at least one point mutation is on one or more residues selected from the group consisting of D835, F691, K663, Y842 and N841). In some embodiments, the at least one point mutation is in the tyrosine kinase domain of FLT3. In some embodiments, the at least one point mutation is in the activation loop of FLT3. In some embodiments, the at least one point mutation is on one or more amino acid residue positions selected from the group consisting of 686, 687, 688, 689, 690, 691, 692, 693, 694, 695, and 696. In some embodiments, the mutated FLT3 has an additional ITD mutation. In some embodiments, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C. In some embodiments, the at least one point mutation is two or more point mutations present on the same allele. In some embodiments, the at least one point mutation is two or more point mutations present on different alleles.

In some embodiments, the present disclosure provides a method of treating cancer in a subject in need thereof, comprising administering to the subject Compound 7 or a pharmaceutically acceptable salt thereof, wherein the subject has a mutant form of IDH1. In some embodiments, the cancer is a hematological malignancy or B cell malignancy. For example, the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), and diffuse large B-cell lymphoma (DLBCL).

In some embodiments, the mutated IDH1 comprises at least one point mutation. In some embodiments, the at least one point mutation is on one or more residues selected from the group consisting of G97D, R100X, R132X, H133Q, and A134D. In some embodiments, the R132X mutation is selected from the group consisting of R132H, R132C, R132L, R132V, R132S and R132G. In some embodiments, the R132X mutation is R132H or R132C. In some embodiments, the R132X mutation is R132H.

In some embodiments, the patient harbors a co-mutation of any of NPM1, FLT3, TET2, CEBPA, DNMT3A, MLL, and combinations thereof.

In some embodiments, Compound 7 inhibits and/or reduces the activity of wild type or mutant Fms-related tyrosine kinase 3 (FLT3) activity or expression in a subject. In some embodiments, FLT3 is a mutant. In some embodiments, the mutated FLT3 comprises at least one point mutation (e.g. the at least one point mutation is on one or more residues selected from the group consisting of D835, F691, K663, Y842 and N841). In some embodiments, the mutated FLT3 is FLT3-ITD.

In some embodiments, the hematological malignancy is leukemia. For example, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma. In some embodiments, the leukemia is acute myeloid leukemia. In some embodiments, the subject has relapsed or refractory acute myeloid leukemia.

In some embodiments, the cancer is myelodysplastic syndromes (MDS) or myeloproliferative neoplasms (MPN).

In some embodiments, the present disclosure provides a method of treating acute myeloid leukemia in a subject in need thereof, comprising administering to the subject Compound 7 or a pharmaceutically acceptable salt thereof, wherein the subject has a mutant form of IDH1. In some embodiments, the subject has relapsed or refractory acute myeloid leukemia.

In one embodiment, the at least one therapeutically active agent in the single pharmaceutical composition and/or combination composition is an anticancer agent.

In a specific embodiment, Compound 7, or a pharmaceutically acceptable salt, ester, solvate and/or prodrug thereof and at least one therapeutically active agent may be formulated into a single pharmaceutical composition and/or combination composition.

In a specific embodiment, the present invention may be a pharmaceutical combination comprising a therapeutically effective amount of Compound 7 or a pharmaceutically acceptable salt, ester, solvate and/or prodrug thereof, and at least one additional anticancer agent. In a specific embodiment, the anticancer agent is a BCL-2 (B-cell lymphoma 2) protein inhibitor. In another specific embodiment, the BCL-2 protein inhibitor is selected from one or more of the group consisting of venetoclax, navitoclax, and ABT-737. In another embodiment, the BCL-2 protein inhibitor is venetoclax.

In another embodiment, the pharmaceutical combination includes Compound 7 and venetoclax both in an oral dosage form. In a specific embodiment, both Compound 7 and venetoclax are in the same oral dosage form. In a specific embodiment, the the oral dosage composition is a tablet.

In another embodiment, Compound 7 and venetoclax are co-administered to a subject.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE FIGURES

FIG. 1 is a volcano plot showing that FLT3-ITD and IDH-1 mutant AML cells from primary patient samples are highly sensitive to Compound 7.

FIG. 2 is a scatter plot showing the IC₅₀ values of compound 7 towards malignant bone marrow or peripheral blood cells from AML patients (118 patients), and with those AML, patents with a mutation in IDH1, a FLT3-ITD mutation and/or IDH2 mutation.

FIG. 3 is a scatter plot showing Area Under the Curve (AUC) values of drug sensitivity of Compound 7 in AML cells from primary patient samples with TP53 wild type or TP53 mutations.

FIG. 4 is a scatter plot showing Area Under the Curve (AUC) values of drug sensitivity of Compound 7 in AML cells from primary patient samples with IDH wild type, IDH1 mutations, IDH2 mutations, SRF2 mutations and IDH2/SRF2 mutations.

FIG. 5 is a scatter plot showing Area Under the Curve (AUC) values of drug sensitivity of Compound 7 in AML cells from primary patient samples with ASXL1 wild type or ASXL1 mutations.

FIG. 6 is a plot showing the IC₅₀ values of compound 7, Venetoclax and the combination of Compound 7 and Venetoclax towards malignant bone marrow or peripheral blood cells from AML patients.

FIG. 7 is a plot showing the IC₅₀ values of compound 7, Venetoclax and the combination of Compound 7 and Venetoclax towards malignant bone marrow or peripheral blood cells from B-cell Cancer patients.

FIG. 8 is a plot showing the IC₅₀ values of compound 7, Venetoclax and the combination of Compound 7 and Venetoclax towards malignant bone marrow or peripheral blood cells from CLL or ALL patients.

FIG. 9 is a plot showing the IC₅₀ values of compound 7, Venetoclax and the combination of Compound 7 and Venetoclax towards malignant bone marrow or peripheral blood cells from AML or MDS/MPN patients.

### DETAILED DESCRIPTION OF THE INVENTION

The present disclosure, the present disclosure provides a method of inhibiting or reducing mutated IDH1 activity or expression in a subject comprising administering Compound 7 or a pharmaceutically acceptable salt, esters, prodrugs, hydrates, solvates and isomers thereof, for the treatment of cancer, such as blood cancers driven by aberrant activation of this gene. Furthermore, in view of the foregoing challenges relating to treating B-cell malignancies associated with mutated IDH1 (e.g., R132H IDH1), Compound 7 was discovered to be more potent against B-cell malignant cell lines (e.g. AML cell lines); more so than conventional IDH1 therapeutic agents (e.g., Tibsovo®). Further, Compound 7 inhibits additional kinases (FLT3, BTK, AURK, c-Src and others) operative in B Cell malignancies.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Compound 7 refers to 1-{3-fluoro-4-[7-(5-methyl-1H-imidazol-2-yl)-1-oxo-2,3-dihydro-1H-isoindol-4-yl]-phenyl}-3-(2,4,6-trifluorophenyl)urea and has the structure below:

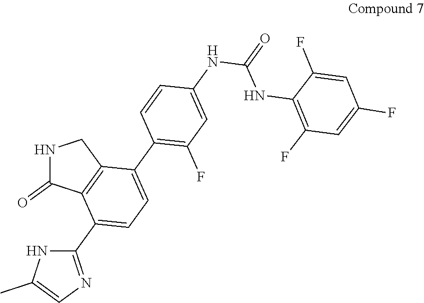 [SMILES: Cc1cnc(-c2ccc(-c3ccc(NC(=O)Nc4c(F)cc(F)cc4F)cc3F)c3c2C(=O)NC3)[nH]1]

The present invention also includes pharmaceutically acceptable salts, esters, prodrugs, hydrates, solvates and isomers thereof, of compound 7.

A “pharmaceutically acceptable salt” includes both acid and base addition salts.

A pharmaceutically acceptable salt of Compound 7 may be a “pharmaceutically acceptable acid addition salt” derived from inorganic or organic acid, and such salt may be pharmaceutically acceptable nontoxic acid addition salt containing anion. For example, the salt may include acid addition salts formed by inorganic acids such as hydrochloric acid, sulfuric acid, nitric acid, phosphoric acid, hydrobromic acid, hydroiodic acid, and the like; organic carbonic acids such as tartaric acid, formic acid, citric acid, acetic acid, trichloroacetic acid, trifluoroacetic acid, gluconic acid, benzoic acid, lactic acid, fumaric acid, maleic acid, and the like; and sulfonic acids such as methanesulfonic acid, benzenesulfonic acid, p-toluenesulfonic acid, naphthalensulfonic acid, and the like.

The pharmaceutically acceptable salt of Compound 7 may be prepared by conventional methods well-known in the art. Specifically, the “pharmaceutically acceptable salt” in accordance of the present invention may be prepared by, e.g., dissolving the Compound 7 in a water-miscible organic solvent such as acetone, methanol, ethanol or acetonitrile and the like; adding an excessive amount of organic acid or an aqueous solution of inorganic acid thereto; precipitating or crystallizing the mixture thus obtained. Further, it may be prepared by further evaporating the solvent or excessive acid therefrom; and then drying the mixture or filtering the extract by using, e.g., a suction filter.

The term “ester” as used herein refers to a chemical moiety having chemical structure of —(R)ₙ—COOR′, wherein R and R′ are each independently selected from the group consisting of alkyl, cycloalkyl, aryl, heteroaryl (connected to oxygen atom by aromatic ring) and heteroalicyclic (connected by aromatic ring), and n is 0 or 1, unless otherwise indicated.

The term “prodrug” as used herein refers to a precursor compound that will undergo metabolic activation in vivo to produce the parent drug. Prodrugs are often useful because they can be easily administered as compared to parent drugs thereof in some cases. For instance, some prodrugs are bioavailable via oral administration unlike parent drugs thereof often show poor bioavailability. Further, the prodrugs may show improved solubility in the pharmaceutical composition as compared to parent drugs thereof. For instance, Compound 7 may be administered in the form of an ester prodrug so as to increase drug delivery efficiency since the solubility of a drug can adversely affect the permeability across the cell membrane. Then, once the compound in the form of the ester prodrug enters a target cell, it may be metabolically hydrolyzed into a carboxylic acid and an active entity.

Hydrates or solvates of Compound 7 are included within the scope of the present invention. As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. The solvent can be water, in which case the solvate can be a hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating particularly pharmaceutically acceptable solvents.

The compounds of the disclosure or their pharmaceutically acceptable salts can contain one or more axes of chirality such that atropisomerization is possible. Atropisomers are stereoisomers arising because of hindered rotation about a single bond, where energy differences due to steric strain or other contributors create a barrier to rotation that is high enough to allow for isolation of individual conformers. The present disclosure is meant to include all such possible isomers, as well as their racemic and optically pure forms whether or not they are specifically depicted herein. Optically active isomers can be prepared using chiral synthons or chiral reagents, or resolved using conventional techniques, for example, chromatography and fractional crystallization. Conventional techniques for the preparation/isolation of individual atropisomers include chiral synthesis from a suitable optically pure precursor or resolution of the racemate (or the racemate of a salt or derivative) using, for example, chiral high pressure liquid chromatography (HPLC).

A “stereoisomer” refers to a compound made up of the same atoms bonded by the same bonds but having different three-dimensional structures, which are not interchangeable. The present invention contemplates various stereoisomers and mixtures thereof as it pertains to atropisomerism.

As used herein, aberrant activation of IDH1 is meant to include divergent, abnormal, atypical, anomalous or irregular IDH1 behavior that leads to a disease, disorder, or condition. Said diseases, disorders, and conditions, may include cancers such as AML, but not limited hereto. In the case of cancer, the disease, disorder, and condition can be characterized by uncontrolled cell proliferation.

Specific examples of diseases associated with IDH1 include but are not limited to glioma, glioblastoma multiforme, paraganglioma, supratentorial primordial neuroectodermal tumors, acute myeloid leukemia (AML), prostate cancer, thyroid cancer, colon cancer, chondrosarcoma, cholangiocarcinoma, peripheral T-cell lymphoma, melanoma, and the like (L. Deng et al., Trends Mol. Med., 2010, 16, 387; T. Shibata et al., Am. J. Pathol., 2011, 178(3), 1395; Gaal et al., J. Clin. Endocrinol. Metab. 2010; Hayden et al., Cell Cycle, 2009; Balss et al., Acta Neuropathol., 2008).

Compound 7 herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor).

When treatment as described above refers to prevention of a disease, disorder, or condition, said treatment is termed prophylactic. Administration of said prophylactic agent can occur prior to the manifestation of symptoms characteristic of a proliferative disorder, such that a disease or disorder is prevented or, alternatively, delayed in its progression.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods, compositions, and combinations of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

As used herein, “polyploidy” or “polyploidy” refers to a condition in which a cell has a number of chromosomes that is some multiple of the monoploid number (“n”) greater than the usual diploid number (“2n”). The term “polyploid cells,” or “polyploidy cells” refers to cells in a polyploidy condition. In other words, the polyploid cell or organism has three or more times the monoploid chromosome number. In humans, the usual monoploid number of chromosomes is 23 and the usual diploid number of chromosomes is 46.

“Mammal” includes humans and both domestic animals such as laboratory animals and household pets (e.g., cats, dogs, swine, cattle, sheep, goats, horses, rabbits), and non-domestic animals such as wildlife and the like. The term “patient” or “subject” as used herein, includes humans and animals.

“Non-mammal” includes a non-mammalian invertebrate and non-mammalian vertebrate, such as a bird (e.g., a chicken or duck) or a fish.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors or slower cell proliferation. Typically, a prophylactic dose is used in subjects prior to or at an earlier stage of disease, so that a prophylactically effective amount can be less than a therapeutically effective amount.

The term “Bruton's tyrosine kinase,” or BTK, as used herein, refers to Bruton's tyrosine kinase from Homo sapiens, as disclosed in, e.g., U.S. Pat. No. 6,326,469 (GenBank Accession No. NP 000052).

The term “covalent BTK inhibitor”, as used herein, refers to an inhibitor that reacts with BTK to form a covalent complex. In some embodiments, the covalent BTK inhibitor is an irreversible BTK inhibitor.

The term “non-covalent BTK inhibitor”, as used herein, refers to an inhibitor that reacts with BTK to form a non-covalent complex or interaction. In some embodiments, the non-covalent BTK inhibitor is a reversible BTK inhibitor.

The terms “pharmaceutical combination,” “therapeutic combination” or “combination” as used herein, refers to a single dosage form comprising at least two therapeutically active agents, or separate dosage forms comprising at least two therapeutically active agents together or separately for use in combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration.

The term “anticancer agents” refers to chemicals and biologics which may treat, reduce, prevent, or ameliorate conditions cause by cancer or tumor growth.

The term “composition” or “formulation” denotes one or more substance in a physical form, such as solid, liquid, gas, or a mixture thereof. One example of composition is a pharmaceutical composition, i.e., a composition related to, prepared for, or used in medical treatment.

The term “co-administration” or “coadministration” refers to administration of Compound 7, or a pharmaceutically acceptable salt, ester, solvate and/or prodrug thereof and (b) at least one additional therapeutically active agent, such as an anticancer agent, together in a coordinated fashion. For example, the co-administration can be simultaneous administration, sequential administration, overlapping administration, interval administration, continuous administration, or a combination thereof.

In one embodiment, the co-administration is carried out for one or more treatment cycles. By “treatment cycle”, it is meant a pre-determined period of time for co-administering the compound of Compound 7, or a pharmaceutically acceptable salt, ester, solvate and/or prodrug thereof and at least one therapeutically active agent. Typically, the patient is examined at the end of each treatment cycle to evaluate the effect of the present combination therapy. In one embodiment, the co-administration is carried out for 1 to 48 treatment cycles. In another embodiment, the co-administration is carried out for 1 to 36 treatment cycles. In another embodiment, the co-administration is carried out for 1 to 24 treatment cycles.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about a month-long treatment cycle. In another embodiment, the treatment cycle has from about 4 to about 6 weeks.

### Methods of Inhibiting IDH1 and Other Mutant Gene Activity

In some embodiments, the present disclosure provides a method of inhibiting or reducing mutated IDH1 activity or expression in a subject comprising administering Compound 7 or a pharmaceutically acceptable salt thereof. In some embodiments, the mutated IDH1 comprises at least one point mutation. For example, the at least one point mutation is on one or more residues selected from the group consisting of G97X, R100X, R132X, H133X, and A134X, wherein X is any amino acid residue. In some embodiments, the G97X mutation is G97D and/or the H133X mutation is H133Q, and/or the A134X mutation is A134D. In some embodiments, the R132X mutation is R132H or R132C. In some embodiments, the R132X mutation is R132H. Thus, in some embodiments, the present disclosure provides a method of inhibiting or reducing mutated IDH1 activity or expression in a subject comprising administering Compound 7 or a pharmaceutically acceptable salt thereof, wherein the mutation is R132H.

In some embodiments, the at least one point mutation is two or more point mutations present on the same allele. In some embodiments, the at least one point mutation is two or more point mutations present on different alleles. In some embodiments, the subject is a mammal. In some embodiments, the mammal is a human.

In some embodiments, the subject harbors a co-mutation of any of NPM1, FLT3, TET2, CEBPA, DNMT3A, MLL, and combinations thereof.

In some embodiments, the subject harbors a mutant form of one or more of IDH1, IDH2, TP53 (tumor protein p53 gene), ASXL1 (additional sex combs like 1) gene, and SRSF2 (Serine/arginine-rich splicing factor 2 gene). In a specific embodiment, the mutations are in the somatic cell of a subject. In another embodiment, the mutations are in one allele. In a specific embodiment, the subject additionally harbors a mutant form of a FLT3. In another specific embodiment, the mutant form of a FLT3 is a tyrosine kinase domain mutation. In another specific embodiment, the mutation is any mutant described in Cancer Cell. 2018 Aug. 13; 34(2): 186-195, which is incorporated by reference herein in its entirety.

In some embodiments, the subject harbors a mutant form of one or more of IDH1, IDH2, and TP53.

In a specific embodiment, the subject harbors a TP53 mutation. In another embodiment, the TP53 mutation is a missense mutation in the somatic cell of the subject. In another embodiment, the mutation is between codons 125 and 300. In another embodiment, wherein the mutation is in the region coding for the DNA binding domain of TP53 gene. In another specific embodiment, the mutation is in one or more codons 175, 248, and 273 of the TP53 gene. In another specific embodiment, the mutation is in one or more codons 196, 213, 245, 282 and 306 of the TP53 gene.

In another embodiment, the gene mutation may be any mutation as described in Cold Spring Harb Perspect Biol. 2010 January; 2(1): a001008, which is incorporated by reference herein in its entirety.

In another embodiment, the gene mutation may be any mutation as described in Nature, 2018 October; 562(7728): 526-531, which is incorporated by reference herein in its entirety.

In another embodiment, the subject harbors a mutation in the ASXL1 gene. In a specific embodiment, the mutation of ASXL1 is from a duplication of a guanine nucleotide (c.1934dupG), otherwise known as NM_015338.5:c.1934dup; p.Gly646Trpfs*12 (ASXL1 c.1934dupG).

In another embodiment, the subject harbors a mutation in the Serine and arginine rich splicing factor 2 (Srsf2) gene. In a specific embodiment, the Srsf2 mutation results in a mutation in amino acid 95 of the protein of Srsf2. In another specific embodiment, the Srsf2 mutation results in amino acid mutation Pro95His, Pro95Leu and P95Arg of the protein of Srsf2. In a specific embodiment, the Srsf2 mutation results in amino acid mutation Pro95His of the protein.

In some embodiments, the methods of the present disclosure further includes inhibiting or reducing wild type or mutant Fms-related tyrosine kinase 3 (FLT3) activity or expression in a subject in need thereof (i.e. a subject having mutated IDH1 activity or expression). FLT3 refers to a protein encoded by the FLT3 gene. Wild-type FLT3 refers to the protein in a non-mutated form. FLT3 can undergo a series of mutations, including the activating internal tandem duplication (ITD) in the juxtamembrane region and point mutations in the tyrosine kinase domain or the activation loop of FLT3. Point mutations occur when a single base pair in a DNA sequence is modified. For instance, F691L is meant to define a change from phenyalanine to leucine for the amino acid at position 691.

In some embodiments, the FLT3 is mutated. For example, in some embodiments, the mutated FLT3 comprises at least one point mutation. In some embodiments, the at least one point mutation is on one or more residues selected from the group consisting of D835, F691, K663, Y842 and N841. Thus, in one embodiment, the at least one point mutation is on D835. In one embodiment, the at least one point mutation is on F691. In one embodiment, the at least one point mutation is on K663. In one embodiments, the at least one point mutation is on Y842. In one embodiments, the at least one point mutation is on N841.

In some embodiments, the at least one point mutation is in the tyrosine kinase domain of FLT3. In some embodiments, the at least one point mutation is in the activation loop of FLT3. In some embodiments, the at least one point mutation is on one or more amino acid residue positions selected from the group consisting of 686, 687, 688, 689, 690, 691, 692, 693, 694, 695, and 696.

In one embodiment, the mutated FLT3 has an additional ITD mutation. In one embodiment, ITD-mutation is associated with very poor prognosis in FTD-driven hematologic cancers, such as AML.

In some embodiments, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C. In some embodiments, the at least one point mutation is two or more point mutations present on the same allele. In some embodiments, the at least one point mutation is two or more point mutations present on different alleles.

In one embodiment of any methods disclosed herein, at least one point mutation is on amino acid residue position 686. In one embodiment, at least one point mutation is on amino acid residue position 687. In one embodiment, at least one point mutation is on amino acid residue position 688. In one embodiment, at least one point mutation is on amino acid residue position 689. In one embodiment, at least one point mutation is on amino acid residue position 690. In one embodiment, at least one point mutation is on amino acid residue position 691. In one embodiment, at least one point mutation is on amino acid residue position 692. In one embodiment, at least one point mutation is on amino acid residue position 693. In one embodiment, at least one point mutation is on amino acid residue position 694. In one embodiment, at least one point mutation is on amino acid residue position 695. In one embodiment, at least one point mutation is on amino acid residue position 696. In another embodiment, the at least one point mutations in on an amino residue that corresponds to position any residues 686-696.

In another embodiment, mutated FLT3 is FLT3-D835H. In another embodiment, mutated FLT3 is FLT3-D835V. In another embodiment, mutated FLT3 is FLT3-D835Y. In another embodiment, mutated FLT3 is FLT3-ITD-D835V. In another embodiment, mutated FLT3 is FLT3-ITD-D835Y. In another embodiment, mutated FLT3 is FLT3-ITD-D835H. In another embodiment, mutated FLT3 is FLT3-ITD-F691L. In another embodiment, mutated FLT3 is FLT3-K663Q. In another embodiment, mutated FLT3 is FLT3-N8411. In another embodiment, mutated FLT3 is FLT3-D835G, FLT3-Y842C, and/or FLT3-ITD-Y842C.

In some embodiments, the present disclosure provides a method of inhibiting or reducing the abnormal (e.g., overexpressed) wild-type or mutated BTK activity or expression in a subject in need thereof (i.e. a subject having mutated IDH1 activity or expression), comprising administering Compound 7 or a pharmaceutically acceptable salt thereof to the subject.

In certain embodiments, the BTK is wild-type. In one embodiment, the wild-type BTK is abnormal (e.g., overexpressed) in a subject. In another embodiment, the wild-type BTK is overactive or hyperactive in a subject.

In certain embodiments, the BTK is mutated BTK. The BTK mutation may be caused by a variety of factors, which are readily apparent to a skilled artisan, such as an insertion mutation, deletion mutation, and substitution mutation (e.g., point mutation). In one embodiment, the mutated BTK comprises at least one point mutation.

A variety of point mutations are contemplated within the scope of the present disclosure. For instance, the at least one point mutation may be to any residue on the BTK. In some embodiments, a mutation within the BTK gene includes a mutation at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, R288, L295, G302, R307, D308, V319, Y334, L358, Y361, H362, H364, N365, 5366, L369, 1370M, R372, L408, G414, Y418, 1429, K430, E445, G462, Y476, M477, C481, C502, C506, A508, M509, L512, L518, R520, D521, A523, R525, N526, V535, L542, R544, Y551, F559, R562, W563, E567, 5578, W581, A582, F583, M587, E589, 5592, G594, Y598, A607, G613, Y617, P619, A622, V626, M630, C633, R641, F644, L647, L652, V1065, and/or A1185. In some embodiments, a mutation within the BTK gene is selected from among L11P, K12R, S14F, K19E, F25S, K27R, R28H, R28C, R28P, T33P, Y3S9, Y40C, Y40N, E41K, I61N, V64F, V64D, R82K, Q103Q5FSSVR, V113D, S115F, T117P, Q127H, C1545, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, 1370M, R372G, L408P, G414R, Y418H, I429N, K430E, E445D, G462D, G462V, Y476D, M477R, C481S, C502F, C502W, C506Y, C506R, A508D, M5091, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M6301, M630K, M630T, C633Y, R641C, F644L, F644S, L647P, L652P, V10651, and A1185V. In one embodiment, the at least one point mutation is on a cysteine residue. In one embodiment, the cysteine residue is in the kinase domain of BTK. In some embodiments, the at least one point mutation is one or more selected from the group consisting of residues E41, P190, and C481. In some embodiments, the mutation in BTK is at amino acid position 481 (i.e., C481). The C481 point mutation may be substituted with any amino acid moiety. In some embodiments, the mutation in BTK is C481S. In one embodiment, the point mutation at residue C481 is selected from C481S, C481R, C481T and/or C481Y. In one embodiment, the at least one point mutation is one or more selected from the group consisting of E41K, P190K, and C481S.

### Methods of Treatment

In some embodiments, the present disclosure provides a method of treating cancer in a subject in need thereof, comprising administering to the subject Compound 7 or a pharmaceutically acceptable salt thereof, wherein the subject has a mutant form of IDH1. In some embodiments, the cancer is a hematological malignancy or B cell malignancy. In some embodiments, the cancer is a B cell malignancy. For example, the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), and diffuse large B-cell lymphoma (DLBCL). In some embodiments, the B cell malignancy is mantle cell lymphoma (MCL). In some embodiments, the B cell malignancy is B-cell acute lymphoblastic leukemia (B-ALL). In some embodiments, the B cell malignancy is Burkitt's lymphoma. In some embodiments, the B cell malignancy is chronic lymphocytic leukemia (CLL). In some embodiments, the B cell malignancy is diffuse large B-cell lymphoma (DLBCL).

In some embodiments, the cancer is a hematological malignancy. Examples of hematological malignancies include, but are not limited to, leukemias, lymphomas, Hodgkin's disease, and myeloma. Also, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), and multiple myeloma (MM).

In some embodiments, the hematological malignancy is leukemia. For example, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiatedvleukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma. In some embodiments, the leukemia is acute myeloid leukemia. In some embodiments, the subject has relapsed or refractory acute myeloid leukemia.

In some embodiment, the cancer is selected from one or more of the group consisting of Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, and Women's Cancers.

In some embodiments, the mutated IDH1 comprises at least one point mutation. In some embodiments, the at least one point mutation is on one or more residues selected from the group consisting of G97D, R100X, R132X, H133Q, and A134D. In some embodiments, the R132X mutation is selected from the group consisting of R132H, R132C, R132L, R132V, R132S and R132G. In some embodiments, the R132X mutation is R132H or R132C. In some embodiments, the R132X mutation is R132H.

In some embodiments, the subject harbors a co-mutation of any of NPM1, FLT3, TET2, CEBPA, DNMT3A, MLL, and combinations thereof.

In some embodiments, the FLT3 is not mutated. In some embodiments, the FLT3 is additionally mutated with IDH1 in a patient. For example, in some embodiments, the mutated FLT3 comprises at least one point mutation. In some embodiments, the at least one point mutation is on one or more residues selected from the group consisting of D835, F691, K663, Y842 and N841. Thus, in one embodiment, the at least one point mutation is on D835. In one embodiment, the at least one point mutation is on F691. In one embodiment, the at least one point mutation is on K663. In one embodiments, the at least one point mutation is on Y842. In one embodiments, the at least one point mutation is on N841.

In some embodiments, the at least one point mutation is in the tyrosine kinase domain of FLT3. In some embodiments, the at least one point mutation is in the activation loop of FLT3. In some embodiments, the at least one point mutation is on one or more amino acid residue positions selected from the group consisting of 686, 687, 688, 689, 690, 691, 692, 693, 694, 695, and 696.

In one embodiment, the mutated FLT3 has an additional ITD mutation. In one embodiment, ITD-mutation is associated with very poor prognosis in FTD-driven hematologic cancers, such as AML.

In some embodiments, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C. In some embodiments, the at least one point mutation is two or more point mutations present on the same allele. In some embodiments, the at least one point mutation is two or more point mutations present on different alleles.

In one embodiment of any methods disclosed herein, at least one point mutation is on amino acid residue position 686. In one embodiment, at least one point mutation is on amino acid residue position 687. In one embodiment, at least one point mutation is on amino acid residue position 688. In one embodiment, at least one point mutation is on amino acid residue position 689. In one embodiment, at least one point mutation is on amino acid residue position 690. In one embodiment, at least one point mutation is on amino acid residue position 691. In one embodiment, at least one point mutation is on amino acid residue position 692. In one embodiment, at least one point mutation is on amino acid residue position 693. In one embodiment, at least one point mutation is on amino acid residue position 694. In one embodiment, at least one point mutation is on amino acid residue position 695. In one embodiment, at least one point mutation is on amino acid residue position 696. In another embodiment, the at least one point mutations in on an amino residue that corresponds to position any residues 686-696.

In another embodiment, mutated FLT3 is FLT3-D835H. In another embodiment, mutated FLT3 is FLT3-D835V. In another embodiment, mutated FLT3 is FLT3-D835Y. In another embodiment, mutated FLT3 is FLT3-ITD-D835V. In another embodiment, mutated FLT3 is FLT3-ITD-D835Y. In another embodiment, mutated FLT3 is FLT3-ITD-D835H. In another embodiment, mutated FLT3 is FLT3-ITD-F691L. In another embodiment, mutated FLT3 is FLT3-K663Q. In another embodiment, mutated FLT3 is FLT3-N8411. In another embodiment, mutated FLT3 is FLT3-D835G, FLT3-Y842C, and/or FLT3-ITD-Y842C.

FLT3 is one of the targets for cancer therapy. Examples of diseases, disorders, and conditions related to aberrant activation of FLT3 include those resulting from over stimulation of FLT3 due to mutations in FLT3, or disorders resulting from abnormally high amount of FLT3 activity due to abnormally high amount of mutations in FLT3. Without bound to any theory, over-activity of FLT3 has been implicated in the pathogenesis of many diseases, including cancers. Cancers affiliated with over-activity of FLT3 include, but are not limited to, myeloproliferative disorders, such as thrombocytopenia, essential thrombocytosis (ET), agnogenic myeloid metaplasia, myelofibrosis (MF), myelofibrosis with myeloid metaplasia (MMM), chronic idiopathic myelofibrosis (UIMF), and polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematological malignancies, including myelodysplasia, multiple myeloma, leukemias, and lymphomas.

In some embodiments, the present disclosure provides a method of treating acute myeloid leukemia in a subject in need thereof, comprising administering to the subject Compound 7 or a pharmaceutically acceptable salt thereof, wherein the subject has a mutant form of IDH1. In some embodiments, the subject has relapsed or refractory acute myeloid leukemia.

In some embodiments, the present disclosure provides a method of treating a disorder in a subject, the method comprising: administering to the subject in need thereof Compound 7, or a pharmaceutically acceptable salt thereof, in an amount sufficient to provide a reduction in blast cells, e.g., leukemic blast cells, e.g., myeloblasts or myeloid blasts, to thereby treat the disorder. In some embodiments, the disorder is an advanced hematologic malignancy, e.g., an advanced hematologic malignancy characterized by the presence of a mutant allele of IDH1. In some embodiments, the advanced hematologic malignancy is characterized by a mutant allele of IDH1, wherein the IDH1 mutation results in a new ability of the enzyme to catalyze the NAPH-dependent reduction of α-ketoglutarate to R(−)-2-hydroxyglutarate (2HG) in a patient. In one embodiment, the mutant IDH1 has an R132X mutation. In one embodiment, the R132X mutation is selected from R132H, R132C, R132L, R132V, R132S and R132G. In another aspect, the R132X mutation is R132H or R132C. In one embodiment, the R132X mutation is R132H.

In some embodiments, the disorder is selected from acute myelogenous leukemia (AML), myelodysplasia syndrome (MDS), myeloproliferative neoplasms (MPN), myeloproliferative neoplasms (MPN), chronic myelomonocytic leukemia (CMML), B-acute lymphoblastic leukemias (B-ALL), B-acute lymphoblastic leukemias (B-ALL), and lymphoma (e.g., T-cell lymphoma), wherein each is characterized by the presence of a mutant allele of IDH1. In some embodiments, the disorder is selected from advanced IDH1 mutation-positive relapsed and/or refractory AML (R/R AML), untreated AML, and MDS.

Treatment methods provide both prophylactic and therapeutic methods for treating a subject at risk or susceptible to developing a cell proliferative disorder driven by mutated IDH1. In one example, the invention provides methods for preventing a cell proliferative disorder related to IDH1, comprising administration of a prophylactically effective amount of Compound 7 or a pharmaceutically acceptable salt thereof or a pharmaceutical composition comprising Compound 7 to a subject in need thereof. In one embodiment, prophylactic treatment can occur prior to the manifestation of symptoms characteristic of the IDH1 driven cell proliferative disorder, such that a disease or disorder is prevented or, alternatively, delayed in its progression.

In one embodiment, the method induces apoptosis of cells expressing mutant IDH1 in a subject in need thereof, comprising administering Compound 7 or a pharmaceutically acceptable salt thereof to the subject.

In one embodiment, the methods of treating cancer include inhibiting or reducing activity or expression of Bruton's Tyrosine Kinase (BTK) in a subject having an IDH1 mutation by administering Compound 7 or a pharmaceutically acceptable salt thereof to the subject.

In certain embodiments, the BTK is wild-type. In one embodiment, the wild-type BTK is abnormal (e.g., overexpressed) in a subject. In another embodiment, the wild-type BTK is overactive or hyperactive in a subject.

In certain embodiments, the BTK is mutated BTK. The BTK mutation may be caused by a variety of factors, which are readily apparent to a skilled artisan, such as an insertion mutation, deletion mutation, and substitution mutation (e.g., point mutation). In one embodiment, the mutated BTK comprises at least one point mutation.

A variety of point mutations are contemplated within the scope of the present disclosure. For instance, the at least one point mutation may be to any residue on the BTK. In some embodiments, a mutation within the BTK gene includes a mutation at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, R288, L295, G302, R307, D308, V319, Y334, L358, Y361, H362, H364, N365, S366, L369, I370M, R372, L408, G414, Y418, 1429, K430, E445, G462, Y476, M477, C481, C502, C506, A508, M509, L512, L518, R520, D521, A523, R525, N526, V535, L542, R544, Y551, F559, R562, W563, E567, S578, W581, A582, F583, M587, E589, S592, G594, Y598, A607, G613, Y617, P619, A622, V626, M630, C633, R641, F644, L647, L652, V1065, and/or A1185. In some embodiments, a mutation within the BTK gene is selected from among L11P, K12R, S14F, K19E, F25S, K27R, R28H, R28C, R28P, T33P, Y3S9, Y40C, Y40N, E41K, I61N, V64F, V64D, R82K, Q103Q5FSSVR, V113D, S115F, T117P, Q127H, C1545, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, 1370M, R372G, L408P, G414R, Y418H, I429N, K430E, E445D, G462D, G462V, Y476D, M477R, C481S, C502F, C502W, C506Y, C506R, A508D, M5091, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M6301, M630K, M630T, C633Y, R641C, F644L, F644S, L647P, L652P, V10651, and A1185V. In one embodiment, the at least one point mutation is on a cysteine residue. In one embodiment, the cysteine residue is in the kinase domain of BTK. In some embodiments, the at least one point mutation is one or more selected from the group consisting of residues E41, P190, and C481. In some embodiments, the mutation in BTK is at amino acid position 481 (i.e., C481). The C481 point mutation may be substituted with any amino acid moiety. In some embodiments, the mutation in BTK is C481S. In one embodiment, the point mutation at residue C481 is selected from C481S, C481R, C481T and/or C481Y. In one embodiment, the at least one point mutation is one or more selected from the group consisting of E41K, P190K, and C481S.

In some embodiments, the B cell lymphoma is characterized by a plurality of cells having a mutant BTK polypeptide. In some embodiments, the mutant BTK polypeptides contain one or more amino acid substitutions that confers resistance to inhibition by a covalent and/or irreversible BTK inhibitor. In some embodiments, the mutant BTK polypeptides contain one or more amino acid substitutions that confers resistance to inhibition by a covalent and/or irreversible BTK inhibitor that covalently binds to cysteine at amino acid position 481 of a wild-type BTK. In some embodiments, the mutant BTK polypeptides contain one or more amino acid substitutions that confers resistance to inhibition by a covalent and/or irreversible BTK inhibitor selected from PCI-32765 (ibrutinib), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) or JTE-051 (Japan Tobacco Inc). In some embodiments, the mutant BTK polypeptides contain one or more amino acid substitutions that confers resistance to inhibition by ibrutinib. In some instances, the plurality of cells comprises at least two cells. In certain embodiments, the BTK mutant contain one or more amino acid substitutions that confers resistance to inhibition by a non-covalent BTK inhibitor. In certain embodiments, the BTK mutant contain one or more amino acid substitutions that confers resistance to inhibition by a reversible BTK inhibitor.

As described above in some embodiments, the modification comprises a substitution or a deletion of the amino acid at amino acid position 481 compared to a wild type BTK. In some embodiments, the modification comprises substitution of the amino acid at position 481 compared to a wild type BTK. In some embodiments, the modification is a substitution of cysteine to an amino acid selected from among leucine, isoleucine, valine, alanine, glycine, methionine, serine, threonine, phenylalanine, tryptophan, lysine, arginine, histidine, proline, tyrosine, asparagine, glutamine, aspartic acid and glutamic acid at amino acid position 481 of the BTK polypeptide. In some embodiments, the modification is a substitution of cysteine to an amino acid selected from among serine, methionine, or threonine at amino acid position 481 of the BTK polypeptide. In some embodiments, the modification is a substitution of cysteine to serine at amino acid position 481 of the BTK polypeptide (“C481S”).

In some embodiments, the mutations in BTK confer resistance in a B cell proliferative disorder to a TEC inhibitor (e.g. ITK inhibitor, BTK inhibitor such as ibrutinib). In some embodiments, C481S mutation in BTK confers resistance in a B cell proliferative disorder to a TEC inhibitor (e.g. ITK inhibitor, BTK inhibitor such as ibrutinib). In some embodiments, the mutations in BTK confer resistance in a B cell proliferative disorder to a covalent BTK inhibitor. In some embodiments, the mutations in BTK confer resistance in a B cell proliferative disorder to ibrutinib and acalabrutinib.

In one embodiment, the activity of mutated BTK is inhibited less by a covalent irreversible BTK inhibitor than the activity of a wild type BTK by a covalent irreversible BTK inhibitor. The covalent irreversible BTK inhibitor may have an IC₅₀ from at least about 1% higher to at least about 1000% higher for the mutated BTK than for the wild type BTK. For example, the covalent irreversible BTK inhibitor may have an IC₅₀ from at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, to at least about 1000% higher for the mutated BTK than for the wild type BTK. In one embodiment, the covalent irreversible BTK inhibitor has an IC₅₀ at least 50% higher for the mutated BTK than for the wild type BTK. In one embodiment, the irreversible covalent BTK inhibitor is ibrutinib and/or acalabrutinib. For example, the irreversible covalent BTK inhibitor is ibrutinib.

In one embodiment, the point mutation is on only one allele of BTK. In another embodiment, the point mutation is on two alleles of BTK. In one embodiment, the point mutation on the cysteine is on only one allele of BTK. In another embodiment, the point mutation on the cysteine is on two alleles of BTK. In one embodiment, the point mutation on C481 is on only one allele of BTK. In another embodiment, the point mutation on C481 is on two alleles of BTK. In one embodiment, the C481S point mutation is on only one allele of BTK. In another embodiment, the C481S point mutation is on two alleles of BTK.

In one embodiment, the subject is a mammal. In one embodiment, the subject is a human.

Another aspect of the present disclosure is directed to a method for treating cancer in a subject in need thereof, comprising administering to a subject in need thereof Compound 7 or a pharmaceutically acceptable salt thereof, wherein the mutant IDH1-containing subject has a mutant form of BTK.

Another aspect of the present disclosure is directed to a method of treating a B cell malignancy in a subject in need thereof, comprising administering to the subject Compound 7 or a pharmaceutically acceptable salt thereof, wherein the subject has a mutant form of IDH1. In one embodiment, the subject has a mutant form of BTK.

In some embodiments, the B cell malignancy is a chronic lymphocytic leukemia (CLL), small lymphocytic lymphoma (SLL), high risk CLL, or a non-CLL/SLL lymphoma. In some embodiments, the B cell proliferative disorder is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, or extranodal marginal zone B cell lymphoma. In some embodiments, the B cell malignancy is acute or chronic myelogenous (or myeloid) leukemia, myelodysplastic syndrome, or acute lymphoblastic leukemia. In some embodiments, the B cell malignancy is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the B cell malignancy is a B cell proliferative disorder that is classified as high-risk. In some embodiments, the B cell malignancy is high risk CLL or high risk SLL.

Accordingly, in one embodiment, the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), and diffuse large B-cell lymphoma (DLBCL). In one embodiment, the treated B cell malignancy is mantle cell lymphoma (MCL). In another embodiment, the treated B cell malignancy is B-cell acute lymphoblastic leukemia (B-ALL). In one embodiment, the treated B cell malignancy is Burkitt's lymphoma. In one embodiment, the treated B cell malignancy is chronic lymphocytic leukemia (CLL). In one embodiment, the treated B cell malignancy is mantle cell lymphoma (MCL). In one embodiment, the treated B cell malignancy is diffuse large B-cell lymphoma (DLBCL).

B-cell malignancies are neoplasms of the blood and encompass, inter alia, non-Hodgkin lymphoma, multiple myeloma, and leukemia. They can originate either in the lymphatic tissues (as in the case of lymphoma) or in the bone marrow (as in the case of leukemia and myeloma), and they all are involved with the uncontrolled growth of lymphocytes or white blood cells. There are many subtypes of B cell proliferative disorders. The disease course and treatment of B cell proliferative disorder is dependent on the B cell proliferative disorder subtype; however, even within each subtype the clinical presentation, morphologic appearance, and response to therapy is heterogeneous.

In some embodiments, Compound 7 inhibits and/or reduces the activity of Aurora kinase. Aurora kinases (Aurora-A, Aurora-B, Aurora-C) are serine/threonine protein kinases that are essential for proliferating cells and have been identified as key regulators of different steps in mitosis and meiosis, ranging from the formation of the mitotic spindle to cytokinesis. Aurora family kinases are critical for cell division, and have been closely linked to tumorigenesis and cancer susceptibility. In various human cancers over-expression and/or up-regulation of kinase activity of Aurora-A, Aurora-B and/or Aurora C has been observed. Over-expression of Aurora kinases correlates clinically with cancer progression and poor survival prognosis. Aurora kinases are involved in phosphorylation events (e.g. phosphorylation of histone H3) that regulate the cell cycle. Dysregulation of the cell cycle can lead to cellular proliferation and other abnormalities.

Thus, in some embodiments, the present disclosure provides a method of treating a patient having an IDH1 mutation and Compound 7 also inhibits and/or reduces the activity of one or more Aurora kinase.

Without being bound by any particular theory, inhibition of BTK and/or Aurora kinase may lead to failure in cytokinesis and abnormal exit from mitosis, which could result in polyploidy cells, cell cycle arrest, and ultimately apoptosis.

Accordingly, in one embodiment, the administration of Compound 7 induces polyploidies. In another embodiment, the administration of Compound 7 induces apoptosis. For example, in one embodiment, a cell is contacted with an effective amount of Compound 7, thereby causing cellular polyploidies and/or cell cycle arrest and/or apoptosis. The cells may be cancer or tumor cells. Accordingly, in one embodiment, the administration of Compound 7 induces apoptosis in cancer and/or tumor cells. In yet another embodiment, the administration of Compound 7 induces apoptosis in cancer and/or tumor cells expressing mutant BTK (e.g., C481S).

In any of the embodiments of the present disclosure, Compound 7 may inhibit and/or reduce the activity or expression of wild type BTK and/or mutant BTK. Accordingly, in some embodiments, Compound 7 inhibits and/or reduces the activity or expression of wild type BTK. In other embodiments, Compound 7 inhibits and/or reduces the activity or expression of mutant BTK. The mutant BTK may comprise at least one point mutation. In one embodiment, the mutant BTK comprises at least one point mutation on a cysteine residue. In one embodiment, the mutant BTK comprises at least one point mutation at residue C481. In one embodiment, the mutant BTK comprises at least a C481S mutation.

A variety of point mutations are contemplated within the scope of the present disclosure and are described above. For instance, the at least one point mutation may be to any residue on the BTK. In one embodiment, the at least one point mutation is on a cysteine residue. In one embodiment, the cysteine residue is in the kinase domain of BTK. In some embodiments, the at least one point mutation is one or more selected from the group consisting of residues E41, P190, and C481. In some embodiments, the mutation in BTK is at amino acid position 481. The C481 point mutation may be substituted with any amino acid moiety. In some embodiments, the mutation in BTK is C481S. In one embodiment, the point mutation at residue C481 is selected from C481S, C481R, C481T and/or C481Y. In one embodiment, the at least one point mutation is one or more selected from the group consisting of E41K, P190K, and C481S.

### Formulations

The effective amount of Compound 7, pharmaceutically acceptable salts, esters, prodrugs, hydrates, solvates and isomers thereof, or a pharmaceutical composition comprising Compound 7 or a pharmaceutically acceptable salt thereof may be determined by one skilled in the art based on known methods.

In one embodiment, a pharmaceutical composition or a pharmaceutical formulation of the present disclosure comprises Compound 7 or a pharmaceutically acceptable salt thereof, and a pharmaceutically acceptable carrier. Pharmaceutically acceptable carrier, diluent or excipient includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microtine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

For the purposes of this disclosure, the pharmaceutical composition of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). Preferably, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, preferably in physiologically compatible buffers such as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

Pharmaceutical formulations for oral administration may include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In one embodiment, the compounds of the present invention may be administered transdermally, such as through a skin patch, or topically. In one aspect, the transdermal or topical formulations of the present invention can additionally comprise one or multiple penetration enhancers or other effectors, including agents that enhance migration of the delivered compound. Preferably, transdermal or topical administration may be used, e.g., in situations in which location specific delivery is desired.

For administration by inhalation, the compounds of the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges of, e.g., gelatin, for use in an inhaler or insufflators may be formulated. These typically contain a powder mix of the compound and a suitable powder base such as lactose or starch. Compositions formulated for parenteral administration by injection, e.g., by bolus injection or continuous infusion, can be presented in unit dosage form e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Formulations for parenteral administration include aqueous solutions or other compositions in water-soluble form.

Suspensions of the active compounds may also be prepared as appropriate oily injection suspensions. Suitable lipophilic solvents or vehicles may include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

As mentioned above, the compositions of the present invention may also be formulated as a reservoir formulation. Such long acting formulations may be administered by implantation (e.g., subcutaneous or intramuscular) or by intramuscular injection. Thus, for example, the inventive compounds may be formulated with suitable polymeric or hydrophobic materials (e.g., an emulsion in an acceptable oil) or ion exchange resins, or as sparingly soluble derivatives, e.g., a sparingly soluble salt.

For any composition used in the present methods of treatment, a therapeutically effective dose can be estimated initially using a variety of techniques well-known in the art. For example, based on information obtained from a cell culture assay, a dose can be formulated in animal models to achieve a circulating concentration range that includes the IC₅₀. Similarly, dosage ranges appropriate for human subjects can be determined, for example, using data obtained from cell culture assays and other animal studies.

A therapeutically effective dose of an agent refers to the amount of the agent that results in amelioration of symptoms or a prolongation of survival in a subject. Toxicity and therapeutic efficacy of such molecules can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD_{50O}/ED₅₀. Agents that exhibit high therapeutic indices are sought.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods well-known in the art, in view of the specifics of a subject's condition.

In addition, the amount of agent or composition administered will be dependent on a variety of factors, including the age, weight, sex, health condition, degree of disease of the subject being treated, the severity of the affliction, the manner of administration, and the judgment of the prescribing physician.

The compound or pharmaceutical compositions of the present disclosure may be manufactured and/or administered in single or multiple unit dose forms.

In a specific embodiment, the present invention provides a pharmaceutical composition and/or combination comprising a therapeutically effective amount of Compound 7, or a pharmaceutically acceptable salt, ester, solvate and/or prodrug thereof, as disclosed herein, as the active ingredient, combined with a pharmaceutically acceptable excipient or carrier. The excipients are added to the formulation for a variety of purposes.

In some embodiments, Compound 7, or a pharmaceutically acceptable salt, ester, solvate and/or prodrug thereof and at least one therapeutically active agent may be formulated into a single pharmaceutical composition and/or combination. In some embodiments, Compound 7, or a pharmaceutically acceptable salt, ester, solvate and/or prodrug thereof and at least one therapeutically active agent are formulated into a separate pharmaceutical composition and/or combination comprising a pharmaceutically acceptable excipient or a carrier.

In one embodiment, the at least one therapeutically active agent in the single pharmaceutical composition and/or combination composition is an anticancer agent.

In a specific embodiment, Compound 7, or a pharmaceutically acceptable salt, ester, solvate and/or prodrug thereof and at least one therapeutically active agent may be formulated into a single pharmaceutical composition and/or combination composition.

In a specific embodiment, the present invention may be a a pharmaceutical combination comprising a therapeutically effective amount of:

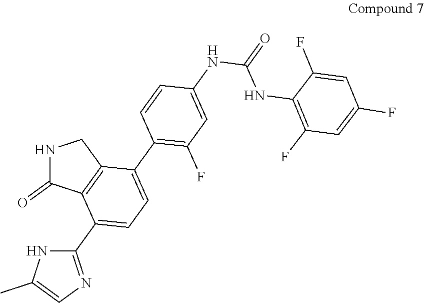 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt or solvate thereof, and at least one additional anticancer agent. In a specific embodiment, the anticancer agent is a BCL-2 (B-cell lymphoma 2) protein inhibitor. In another specific embodiment, the BCL-2 protein inhibitor is selected from one or more of the group consisting of venetoclax, navitoclax, and ABT-737. In another embodiment, the BCL-2 protein inhibitor is venetoclax.

In another embodiment, the pharmaceutical combination includes Compound 7 and venetoclax both in an oral dosage form. In a specific embodiment, both Compound 7 and venetoclax are in the same oral dosage form. In a specific embodiment, the oral dosage composition is a tablet.

In another embodiment, Compound 7 and venetoclax are co-administered to a subject.

In a specific embodiment, the dosage amount of venetoclax is in the range of about 1 mg to about 150 mg. In a specific embodiment, the range is between about 10 and 125 mg. In a specific embodiment, the range is between about 10 and 100 mg. In a specific embodiment, the range is between about 20 and 75 mg. In a specific embodiment, the range is between about 30 and 70 mg.

In a specific embodiment, the dosage amount of Compound 7 is in the range of about 1 mg to about 500 mg. In a specific embodiment, the range is between about 10 and 300 mg. In a specific embodiment, the range is between about 20 and 200 mg. In a specific embodiment, the range is between about 30 and 150 mg. In a specific embodiment, the range is between about 50 and 100 mg.

The active ingredient and excipients may be formulated into compositions and/or combinations and dosage forms according to methods known in the art.

In one embodiment, a dosage form may be provided as a kit comprising Compound 7, or a pharmaceutically acceptable salt, ester, solvate and/or prodrug thereof and pharmaceutically acceptable excipients and carriers as separate components. In one embodiment, a dosage form may be provided as a kit comprising Compound 7, or a pharmaceutically acceptable salt, ester, solvate and/or prodrug thereof, at least one additional therapeutically active agent, and pharmaceutically acceptable excipients and carriers as separate components. In some embodiments, the dosage form kit allow physicians and patients to formulate an oral solution or injection solution prior to use by dissolving, suspending, or mixing the compound of Compound 7, or a pharmaceutically acceptable salt, ester, solvate and/or prodrug thereof with pharmaceutically acceptable excipients and carriers.

Having now generally described the invention, the same will be more readily understood through reference to the following examples, which are provided by way of illustration and are not intended to be limiting of the present invention. Unless expressly stated otherwise, conditions and procedures performed as generally known in the art.

### SPECIFIC EMBODIMENTS OF THE PRESENT INVENTION

### Embodiment 1

A method of inhibiting or reducing mutated IDH1 activity or expression in a subject comprising administering

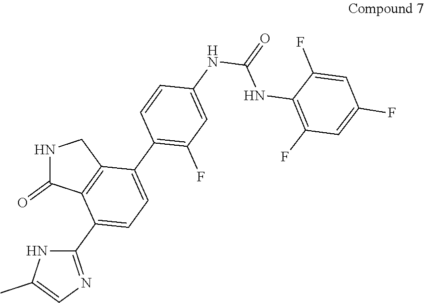 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt thereof.

### Embodiment 2

The method of Embodiment 1, wherein the mutated IDH1 comprises at least one point mutation.

### Embodiment 3

The method of Embodiment 2, wherein the at least one point mutation is on one or more residues selected from the group consisting of G97X, R100X, R132X, H133X, and A134X.

### Embodiment 4

The method of Embodiment 3, wherein the G97X mutation is G97D and/or the H133X mutation is H133Q, and/or the A134X mutation is A134D.

### Embodiment 5

The method of claim Embodiment, wherein the R132X mutation is selected from the group consisting of R132H, R132C, R132L, R132V, R132S and R132G.

### Embodiment 6

The method of Embodiment 5, wherein the R132X mutation is R132H or R132C.

### Embodiment 7

The method of Embodiment 7, wherein the R132X mutation is R132H.

### Embodiment 8

The method of any of preceding Embodiments, wherein the at least one point mutation is two or more point mutations present on the same allele.

### Embodiment 9

The method of any of Embodiment 1-7, wherein the at least one point mutation is two or more point mutations present on different alleles.

### Embodiment 10

The method of any of the preceding claims, wherein the subject is a mammal.

### Embodiment 11

The method of Embodiment 10, wherein the subject is a human.

### Embodiment 12

The method of any of the preceding Embodiments, wherein the method further includes inhibiting or reducing wild type or mutant Fms-related tyrosine kinase 3 (FLT3) activity or expression in a subject in need thereof.

### Embodiment 13

The method of Embodiment 12, wherein the FLT3 is mutated.

### Embodiment 14

The method of Embodiment 13, wherein the mutated FLT3 comprises at least one point mutation.

### Embodiment 15

The method of Embodiment 14, wherein the at least one point mutation is on one or more residues selected from the group consisting of D835, F691, K663, Y842 and N841.

### Embodiment 16

The method of Embodiment 14, wherein the mutated FLT3 comprises at least one mutation at D835.

### Embodiment 17

The method of Embodiment 14, wherein the mutated FLT3 comprises at least one mutation at F691.

### Embodiment 18

The method of Embodiment 14, wherein the mutated FLT3 comprises at least one mutation at K663.

### Embodiment 19

The method of Embodiment 14, wherein the mutated FLT3 comprises at least one mutation at N841.

### Embodiment 20

The method of Embodiment 14, wherein the at least one point mutation is in the tyrosine kinase domain of FLT3.

### Embodiment 21

The method of Embodiment 14, wherein the at least one point mutation is in the activation loop of FLT3.

### Embodiment 22

The method of Embodiment 14, wherein the at least one point mutation is on one or more amino acid residue positions selected from the group consisting of 686, 687, 688, 689, 690, 691, 692, 693, 694, 695, and 696.

### Embodiment 23

The method of Embodiment 14, wherein the mutated FLT3 has an additional ITD mutation.

### Embodiment 24

The method of Embodiment 14, wherein the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C.

### Embodiment 25

The method of Embodiment 22, wherein the at least one point mutation is two or more point mutations present on the same allele.

### Embodiment 26

The method of Embodiment 22, wherein the at least one point mutation is two or more point mutations present on different alleles.

### Embodiment 27

A method of treating cancer in a subject in need thereof, comprising administering to the subject Compound 7:

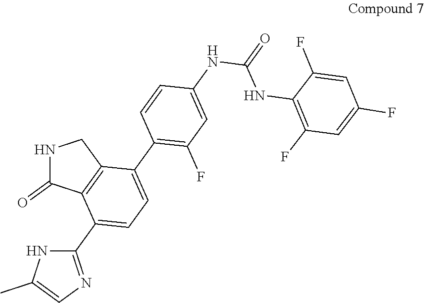 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt thereof, wherein the subject has a mutant form of IDH1.

### Embodiment 28

The method of Embodiment 27, wherein the cancer is a hematological malignancy or B cell malignancy.

### Embodiment 29

The method of Embodiment 28, wherein the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), and diffuse large B-cell lymphoma (DLBCL).

### Embodiment 30

The method of Embodiment 31, wherein the treated B cell malignancy is mantle cell lymphoma (MCL).

### Embodiment 31

The method of Embodiment 31, wherein the treated B cell malignancy is B-cell acute lymphoblastic leukemia (B-ALL).

### Embodiment 32

The method of Embodiment 31, wherein the treated B cell malignancy is Burkitt's lymphoma.

### Embodiment 33

The method of Embodiment 31, wherein the treated B cell malignancy is chronic lymphocytic leukemia (CLL).

### Embodiment 34

The method of Embodiment 31, wherein the treated B cell malignancy is diffuse large B-cell lymphoma (DLBCL).

### Embodiment 35

The method of Embodiment 27, wherein Compound 7 inhibits and/or reduces the activity or expression of mutant IDH1.

### Embodiment 36

The method of Embodiment 35, wherein the mutated IDH1 comprises at least one point mutation.

### Embodiment 37

The method of Embodiment 36, wherein the at least one point mutation is on one or more residues selected from the group consisting of G97D, R100X, R132X, H133Q, and A134D.

### Embodiment 38

The method of Embodiment 37, wherein the R132X mutation is selected from the group consisting of R132H, R132C, R132L, R132V, R132S and R132G.

### Embodiment 39

The method of Embodiment 38, wherein the R132X mutation is R132H or R132C.

### Embodiment 40

The method of Embodiment 39, wherein the R132X mutation is R132H.

### Embodiment 41

The method of any one of Embodiments 27-40, wherein the patient harbors a co-mutation of any of NPM1, FLT3, TET2, CEBPA, DNMT3A, MLL, and combinations thereof.

### Embodiment 42

The method of any one of Embodiments 27-41, wherein Compound 7 inhibits and/or reduces the activity of wild type or mutant Fms-related tyrosine kinase 3 (FLT3) activity or expression in a subject.

### Embodiment 43

The method of Embodiment 42 wherein FLT3 is mutant.

### Embodiment 44

The method of Embodiment 43, wherein the mutated FLT3 comprises at least one point mutation.

### Embodiment 45

The method of Embodiment 44, wherein the at least one point mutation is on one or more residues selected from the group consisting of D835, F691, K663, Y842 and N841.

### Embodiment 46

The method of Embodiment 43, wherein the mutated FLT3 is FLT3-ITD.

### Embodiment 47

The method of Embodiment 28, wherein the hematological malignancy is leukemia.

### Embodiment 48

The method of Embodiment 47, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma.

### Embodiment 49

The method of Embodiment 48, wherein the leukemia is acute myeloid leukemia.

### Embodiment 50

The method of Embodiment 49, wherein the subject has relapsed or refractory acute myeloid leukemia.

### Embodiment 51

A method of treating acute myeloid leukemia in a subject in need thereof, comprising administering to the subject Compound 7:

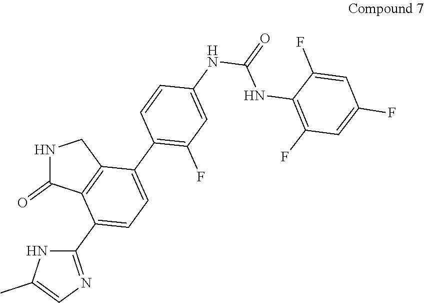 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt thereof, wherein the subject has a mutant form of IDH1.

### Embodiment 52

The method of Embodiment 50, wherein the subject has relapsed or refractory acute myeloid leukemia.

### Embodiment 53

The method of Embodiment 52 or 53, wherein the mutated IDH1 comprises at least one point mutation.

### Embodiment 54

The method of Embodiment 53, wherein the mutation is at least one point mutation is on one or more residues selected from the group consisting of G97, R100X, R132X, H133X, and A134X.

### Embodiment 55

The method of Embodiment 54, wherein the G97X mutation is G97D and/or the H133X mutation is H133Q, and/or the A134X mutation is A134D.

### Embodiment 56

The method of Embodiment 54, wherein the R132X mutation is selected from the group consisting of R132H, R132C, R132L, R132V, R132S and R132G.

### Embodiment 57

The method of Embodiment 56, wherein the R132X mutation is R132H or R132C.

### Embodiment 58

The method of Embodiment 56, wherein the R132X mutation is R132H.

### Embodiment 59

The method of any of Embodiments 53-58, wherein the mutation is at least one point mutation is two or more point mutations present on the same allele.

### Embodiment 60

The method of any of Embodiments 53-58, wherein the mutation is at least one point mutation is two or more point mutations present on different alleles.

### Embodiment 61

The method of any of Embodiments 51-60, wherein the subject is a mammal.

### Embodiment 62

The method of Embodiment 61, wherein the subject is a human.

### Embodiment 63

The method of any one of Embodiments 51-62, wherein the patient harbors a co-mutation of any of NPM1, FLT3, TET2, CEBPA, DNMT3A, MLL, and combinations thereof.

### Embodiment 64

The method of Embodiment 63, wherein FLT3 is a mutant.

### Embodiment 65

The method of Embodiment 64, wherein the mutated FLT3 comprises at least one point mutation.

### Embodiment 66

The method of Embodiment 65, wherein the mutation is at least one point mutation is on one or more residues selected from the group consisting of D835, F691, K663, Y842 and N841.

### Embodiment 67

The method of Embodiment 64, wherein the mutated FLT3 is FLT3-ITD.

### Embodiment 68

The method of any Embodiments 51-67, wherein the subject has a mutation on BTK.

### Embodiment 69

The method of Embodiment 68, wherein the mutation is at least one point mutation.

### Embodiment 70

The method of Embodiment 69, wherein the point mutation is on a cysteine residue and is in the kinase domain of BTK.

### Embodiment 71

The method of Embodiment 68, wherein, the mutation is at least one point mutation is one or more selected from the group consisting of residues E41, P 190, and C481.

### Embodiment 72

The method of Embodiment 68, wherein the mutation in BTK is at amino acid position 481.

### Embodiment 73

The method of Embodiment 72, wherein the mutation in BTK is selected from C481S, C481R, C481T and/or C481Y.

### Embodiment 74

The method of Embodiment 72, wherein the mutation is at least one point mutation is C481S.

### Embodiment 75

The method of any one of Embodiments 27 and 35-46, wherein the cancer is selected from the group consisting of glioma, glioblastoma multiforme, paraganglioma, supratentorial primordial neuroectodermal tumors, prostate cancer, thyroid cancer, colon cancer, chondrosarcoma, cholangiocarcinoma, peripheral T-cell lymphoma, and melanoma.

### Embodiment 76

The method of Embodiment 75, wherein the cancer is selected from glioma chondrosarcoma, and cholangiocarcinoma.

### Embodiment 77

The method of any Embodiments 27-50 and 75-76, wherein the subject has at least one mutation on BTK.

### Embodiment 78

The method of Embodiment 77, wherein the mutation is at least one point mutation.

### Embodiment 79

The method of Embodiment 78, wherein the point mutation is on a cysteine residue and is in the kinase domain of BTK.

### Embodiment 80

The method of Embodiment 78, wherein, the mutation is at least one point mutation is one or more selected from the group consisting of residues E41, P190, and C481.

### Embodiment 81

The method of Embodiment 78, wherein the mutation in BTK is at amino acid position 481.

### Embodiment 82

The method of Embodiment 81, wherein the mutation in BTK is selected from C481S, C481R, C481T and/or C481Y.

### Embodiment 83

The method of Embodiment 82, wherein the mutation is at least one point mutation is C481S.

### Embodiment 84

A method of treating cancer in a subject in need thereof, comprising administering to the subject Compound 7:

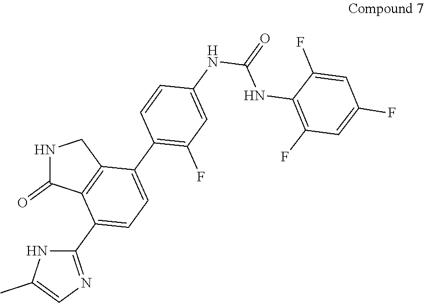 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt thereof, wherein the subject has a mutant form of one or more of IDH1, IDH2, TP53, ASXL1, and SRSF2.

### Embodiment 85

The method of Embodiment 84, wherein the subject additionally has a mutant form of a FLT3.

### Embodiment 86

The method of Embodiment 85, wherein the mutant form of a FLT3 is a tyrosine kinase domain mutation.

### Embodiment 86

The method of Embodiment 84, wherein the subject has a mutant form of one or more of IDH1, IDH2, and TP53.

### Embodiment 87

The method of Embodiment 86, wherein the TP53 mutation is a missense mutation in the somatic cell of the subject.

### Embodiment 88

The method of Embodiment 87, wherein the mutation is between codons 125 and 300.

### Embodiment 89

The method of Embodiment 87, wherein the mutation is in the region coding for the DNA binding domain of TP53 gene.

### Embodiment 90

The method of Embodiment 87, wherein the mutation is in codons 175, 248, and 273 of the TP53 gene.

### Embodiment 91

The method of Embodiment 87, wherein the mutation is in codons 196, 213, 245, 282 and 306 of the TP53 gene.

### Embodiment 92

The method of Embodiment 84, wherein the mutation is of the ASXL1 gene.

### Embodiment 93

The method of Embodiment 92, wherein the mutation of ASXL1 is from a duplication of a guanine nucleotide (c.1934dupG).

### Embodiment 94

The method of Embodiment 84, wherein the mutation is in Serine and arginine rich splicing factor 2 (SRSF2).

### Embodiment 95

The method of Embodiment 94, wherein the Srsf2 mutation results in a mutation in amino acid 95 of the protein.

### Embodiment 96

The method of Embodiment 95, wherein the Srsf2 mutation results in amino acid mutation Pro95His, Pro95Leu and P95Arg of the protein.

### Embodiment 97

The method of Embodiment 96, wherein the Srsf2 mutation results in amino acid mutation Pro95His of the protein.

### Embodiment 98

The method of any one of Embodiments 84-97, wherein the cancer is a hematological malignancy or B cell malignancy.

### Embodiment 99

The method of Embodiment 98, wherein the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), and diffuse large B-cell lymphoma (DLBCL).

### Embodiment 100

The method of Embodiment 99, wherein the treated B cell malignancy is mantle cell lymphoma (MCL).

### Embodiment 100

The method of Embodiment 99, wherein the treated B cell malignancy is B-cell acute lymphoblastic leukemia (B-ALL).

### Embodiment 101

The method of Embodiment 99, wherein the treated B cell malignancy is Burkitt's lymphoma.

### Embodiment 102

The method of Embodiment 99, wherein the treated B cell malignancy is chronic lymphocytic leukemia (CLL).

### Embodiment 103

The method of Embodiment 99, wherein the treated B cell malignancy is diffuse large B-cell lymphoma (DLBCL).

### Embodiment 104

The method of Embodiment 99, wherein the cancer is a hematological malignancy.

### Embodiment 105

The method of any one of Embodiments 84-97, wherein the hematological malignancy is leukemia.

### Embodiment 106

The method of Embodiment 105, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, myelodysplastic syndromes (MDS), myeloproliferative neoplasms (MPN) and/or mantle cell lymphoma.

### Embodiment 107

The method of Embodiment 106, wherein the leukemia is acute myeloid leukemia.

### Embodiment 108

The method of Embodiment 106, wherein the subject has relapsed or refractory acute myeloid leukemia.

### Embodiment 109

The methods of any of Embodiments 1-108, wherein Compound 7 is in a pharmaceutical combination comprising a therapeutically effective amount of:

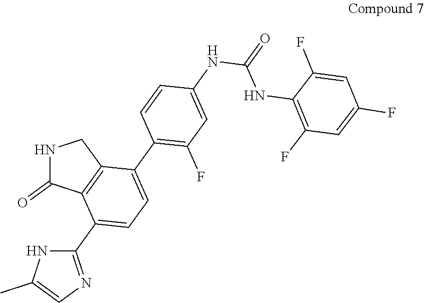 [SMILES: Cc1cnc(-c2ccc(-c3ccc(NC(=O)Nc4c(F)cc(F)cc4F)cc3F)c3c2C(=O)NC3)[nH]1]

or a pharmaceutically acceptable salt or solvate thereof, and at least one additional anticancer agent.

### Embodiment 110

The methods of Embodiment 109, wherein the anticancer agent is a BCL-2 (B-cell lymphoma 2) protein inhibitor.

### Embodiment 111

The methods of Embodiment 110, wherein the BCL-2 protein inhibitor is selected from one or more of the group consisting of venetoclax, navitoclax, and ABT-737.

### Embodiment 112

The methods of Embodiment 111, wherein the combination is Compound 7 and venetoclax.

### EXAMPLES

### Synthesis: Material and Methods

Various starting materials may be prepared in accordance with conventional synthetic methods well-known in the art. Some of the starting materials are commercially available from manufacturers and suppliers of reagents, such as Aldrich, Sigma, TCI, Wako, Kanto, Fluorchem, Acros, Abocado, Alfa, Fluka, etc., but not limited thereto.

The compounds of the present disclosure can be prepared from readily available starting materials by conventional methods and processes below. Different methods may also be used for manufacturing the inventive compounds, unless otherwise specified as typical or optimal process conditions (i.e., reaction temperature, time, molar ratio of reactants, solvents, pressures, etc.). The optimal reaction conditions may vary depending on the particular reactants or solvents employed. Such conditions, however, can be determined by the skilled in the art by conventional optimization process.

In addition, those of ordinary skill in the art recognize that some functional groups can be protected/deprotected using various protecting groups before a certain reaction takes place. Suitable conditions for protecting and/or deprotecting specific functional group, and the use of protecting groups are well-known in the art.

For example, various kinds of protecting groups are described in T. W. Greene and G. M. Wuts, Protecting Groups in Organic Synthesis, Second edition, Wiley, New York, 1991, and other references cited above.

In one embodiment of the present invention, Compound 7 of the present invention may be prepared by synthesizing an intermediate, Compound D, according to the Scheme 1 as shown below, and then subjecting Compound D through the procedure of Reaction Scheme 2. However, the method for synthesizing Compound D above is not limited to Reaction Scheme 1.

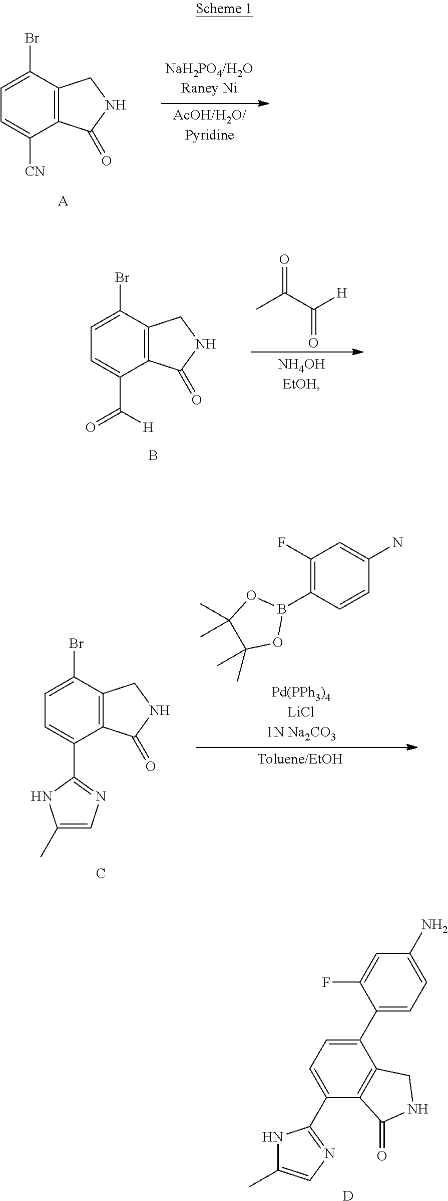

The method for preparing the starting material of Reaction Scheme 1, i.e., Compound

A, is described in International Patent Publication WO2012/014017, and the preparation of Compound D is described in U.S. Patent Application Publication US2015/0336934

### Example 1: Synthesis of 1-{3-fluoro-4-[7-(5-methyl-1H-imidazol-2-yl)-1-oxo-2,3-dihydro-1H-isoindol-4-yl]-phenyl}-3-(2,4,6-trifluoro-phenyl)-urea (Compound 7)

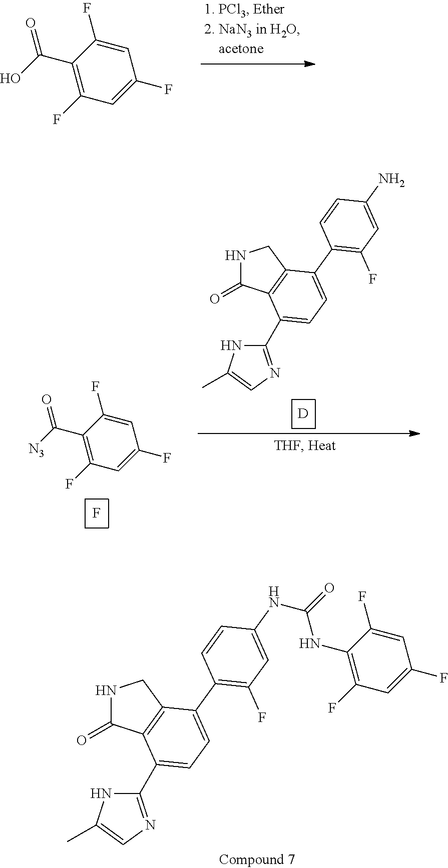

2,4,6-trifluorobenzoic acid (0.08 g, 0.45 mmol) was dispersed in diethyl ether (5.7 mL), slowly added with phosphorus pentachloride (PCl₅, 0.11 g, 0.52 mmol), and then stirred for 1 hour. Upon completion of the reaction, the organic solvent was concentrated under reduced pressure below room temperature, and then the reaction solution was diluted by adding acetone (3.8 mL). Subsequently, sodium azide (NaN₃, 0.035 g, 0.545 mmol) dissolved in water (0.28 mL) was slowly added to the reaction solution dropwise at 0° C. After stirring for 2 hours at room temperature, 2,4,6-trifluorobenzoyl azide thus formed was diluted with ethyl acetate, and then washed with water. The organic layer was dried over anhydrous magnesium sulfate, dispersed in THF (2 mL), added with THF (7.5 mL) containing 4-(4-amino-2-fluorophenyl)-7-(5-methyl-1H-imidazol-2-yl)isoindolin-1-one (Compound D, 0.073 g, 0.23 mmol), and then stirred for 3 hours at 90° C. Upon completion of the reaction, the solvent was concentrated under reduced pressure, and then purified by silica gel column chromatography (eluent:methylene chloride:methanol=20:1) to obtain Compound 7 (0.026 g, yield: 23%). (300 MHz, DMSO-d): 14.46-14.37 (m 1H), 9.47-9.45 (br m, 1H), 9.37 (s, 1H), 8.45 (d, J=1.8 Hz, 1H), 8.30-8.27 (br m, 1H), 7.63-7.46 (m, 3H), 7.31-7.26 (m, 3H), 7.09-6.84 (m, 1H), 4.42 (s, 2H), 2.31-2.21 (m, 3H). LCMS [M+1]: 496.3.

### Genomics Analysis Examples

For Genomics Analysis examples 2-4 below, studies were performed according to published procedures [Tyner, J. W., Tognon, C. E., Bottomly, D. et al. Functional genomic landscape of acute myeloid leukaemia. Nature 562, 526-531 (2018) doi:10.1038/s41586-018-0623-z; Kurtz, S. E., Eide, C. A., Kaempf, A. et al. Molecularly targeted drug combinations demonstrate selective effectiveness for myeloid- and lymphoid-derived hematologic malignancies. PNAS Sep. 5, 2017 114 (36) 7554-7563], which are incorporated by reference in their entirety herein.

All patient samples were analyzed for clinical characteristics and drug sensitivity. AML, CLL, ALL and MDS/MPN and other patient samples were analyzed with respect to expanded, disease-specific panels of clinical, prognostic, genetic, cytogenetic, and surface antigen characteristics obtained from patient electronic medical records. Genetic characterization of AML samples included results of a clinical deep-sequencing panel of genes commonly mutated in hematologic malignancies (GeneTrails panel from Knight Diagnostic Laboratories, OHSU; Foundation Medicine reports from UT Southwestern).

Compound 7 and/or venetoclax were prepared in a well in a seven-point concentration series. Similar plates were prepared with the 48 indicated pairwise inhibitor combinations in seven-point fixed molar concentration series identical to those used for single agents. The final concentration of DMSO was ≤0.1% in all wells, and all sets of single-agent and combination destination plates were stored at −20° C. and thawed immediately before use. Primary mononuclear cells were plated across single-agent and combination inhibitor panels within 24 h of collection. Cells were seeded into assay plates at 10,000 cells per well in RPMI 1640 media supplemented with FBS (10%), L-glutamine, penicillin/streptomycin, and β-mercaptoethanol (10⁻⁴ M). After 3 d of culture at 37° C. in 5% CO₂, MTS reagent (CellTiter96 AQᵤₑₒᵤₛ One; Promega) was added, optical density was measured at 490 nm, and raw absorbance values were adjusted to a reference blank value and then used to determine cell viability (normalized to untreated control wells).

Normalized viability values at each dose of a seven-point dilution series for Compound 7, venetoclax or combinations of the two compounds were analyzed for each of numerous primary leukemia samples. Dose concentrations were log 10-transformed, and a probit regression curve was fit to each seven-point drug sensitivity profile by using maximum-likelihood estimation for the intercept and slope. This parametric model was chosen over a polynomial because the probit's monotonic shape reflects a dose-response curve typically seen in samples incubated with cytotoxic or inhibitory agents. Normalized viability values greater than 100%, indicating higher cell viability than the average viability across control wells on a given plate, were truncated to 100% to produce a percentage response variable amenable to probit modeling. From the fitted probit curve for each sample/drug pairing, the IC₅₀ was defined as the lowest concentration to achieve 50% predicted viability and the AUC was computed by integration of the curve height across the tested dose range. If the predicted cell viability (i.e., probit curve height) was ≤50% at the lowest tested dose or >50% across the entire dose range, the IC₅₀ was designated as the lowest dose or highest dose, respectively. For sensitivity profiles with 100% normalized viability at all seven dose points, the IC₅₀ and AUC were designated as the highest tested dose and the maximum possible AUC, respectively. For sensitivity profiles with 0% viability at all seven dose points, the IC₅₀ and AUC were designated as the lowest tested dose and a value (0.5) just below the minimum probit-derived AUC, respectively.

To quantify the efficacy of an equimolar drug combination in comparison with its constituent single agents, a CR effect measure was generated based on the specific IC₅₀ and AUC values for each inhibitor triad (the drug combination and the two single agents). The IC₅₀ CR and AUC CR values were defined as the ratio of the combination's IC₅₀ or AUC to the minimum IC₅₀ or AUC for the two single agents, respectively. Each sensitivity profile modeled by probit regression was assigned a fit statistic based on the P value for the test of whether the fitted curve's slope was horizontal. Generally, a smaller fit statistic produced by a decreasing slope indicates a better fit and, by extension, provides a measure of confidence in the curve-derived IC₅₀ and AUC for a particular sample/drug pair. A CR effect measure value less than 1 indicates that a sample is more sensitive to the drug combination than it is to either of the single agents that constitute the combination.

### Example 2: Genomics Analysis of Compound 7

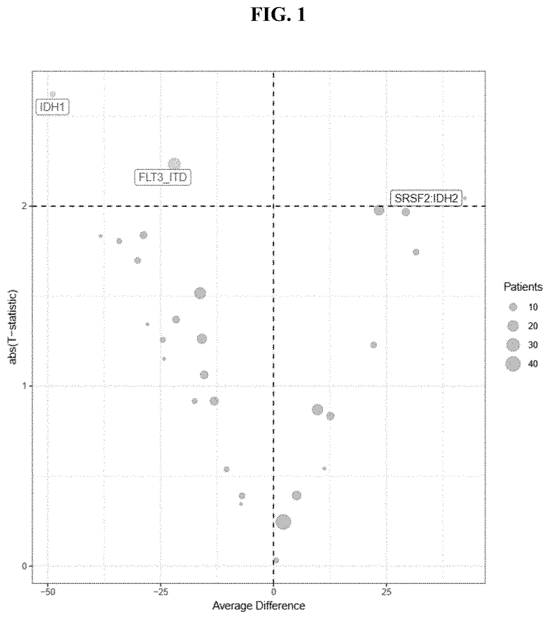

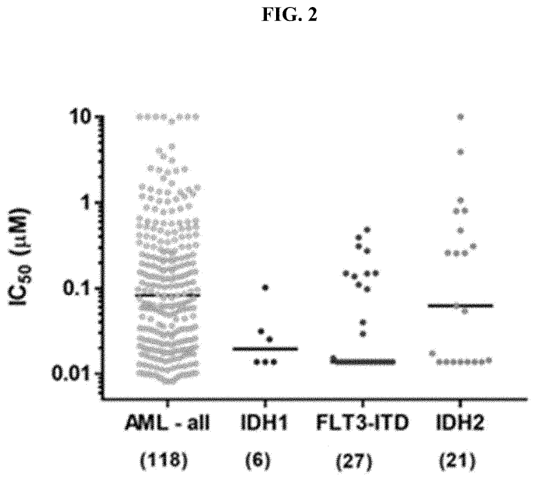

Genomics analysis of Compound 7 was performed by testing the association of somatic mutations and expression data with Compound 7 response on freshly harvested malignant bone marrow or peripheral blood cells from AML patients. Drug sensitivity with clinical status, gene abnormalities and gene expression levels, whole exome sequencing (n=118) and RNA sequencing were performed. Patient samples with FLT3-ITD mutations were more sensitive to compound 7 as compared to wild-type (WT). Furthermore, patient samples with IDH1 mutants demonstrated greater sensitivity to compound 7 relative to WT. Most unexpectedly, on average the patient samples with IDH1 R132 residue mutants (n==6) demonstrated significantly greater sensitivity to compound 7 relative to WT. This association is indicated in the Volcano Plot of FIG. 1, which shows the mutated sensitivity level of a given test (Y-axis) in conjunction with its estimated effect (X-axis), and the Scatter Plot of FIG. 2, which illustrated the individual IC₅₀ of compound 7 on each patient sample. FIG. 2 shows the compound 7 IC₅₀ towards killing cells in each AML patient bone morrow sample. As shown in FIG. 2, the IC₅₀ is particular low for all patient samples with IDH1 mutations, indicating compound 7 is particularly effective at treating malignant cells with IDH1 mutations. FIG. 2 also indicates compound 7 effectiveness of patient samples with FLT3-ITD mutations.

### Example 3: Genomics Analysis of Compound 7

Genomics analysis of Compound 7 was performed by testing the association of somatic mutations and expression data with compound 7 response on various cancer cell lines. Inhibitor activity was assessed by an ex vivo assay to determine sensitivities of drugs on freshly isolated primary patient samples. Cell viability was assessed after 72-hour culture using a tetrazolium-based MTS assay and IC50 and Area Under the Curve (AUC) values calculated as a measure of drug sensitivity. Under the culture conditions used here, the cells retain viability (>90%), but do not proliferate.

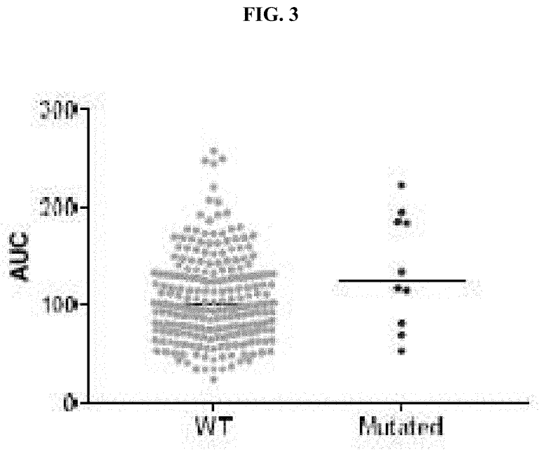

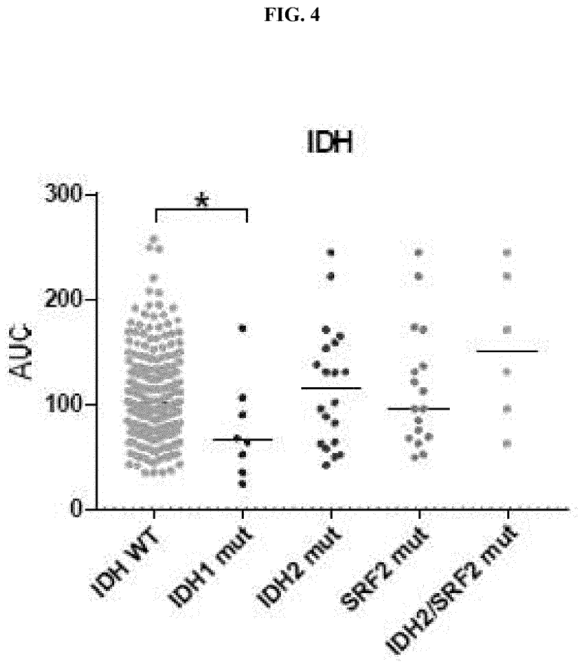

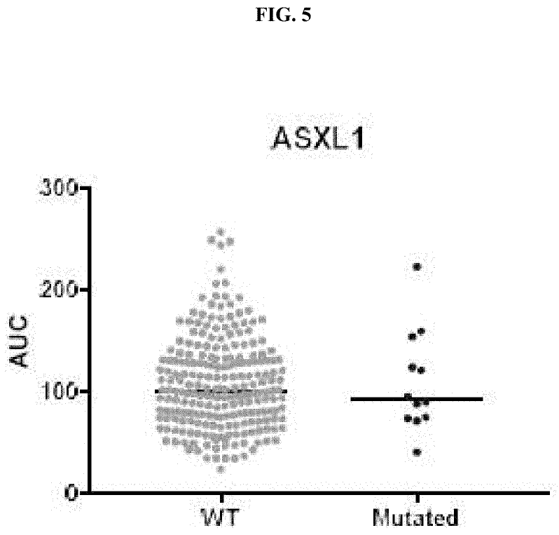

The Assay indicates that compound 7 is particularly effective at treating malignant cells with specific mutations. Although AML cancer cells with TP53 mutations are generally less sensitive to various drugs relative to AML cells with wild type TP53, FIG. 3 demonstrates in the scatter plot that Compound 7 retains effectiveness in treating AML cells with TP53 mutations. FIG. 4 indicates that Compound 7 is substantially more effective in treating AML cells with IDH mutations compared to IDH wild type AML cells. FIG. 4 also shows that compound 7 is just as effective against cancers with SRSF2 mutations as with wild type. This is important as many other drugs, such as sunitinib and crenolanib appear resistant to SRSF2 mutant cells. Similarly, FIG. 5 also shows that compound 7 is just as effective against cancers with ASXL1 mutations as with wild type in AML cells. This again is important as many other drugs, such as sunitinib and crenolanib appear resistant to ASXL1 mutant cells.

### Example 4: Genomics Analysis of Compound 7 in Combination with Venetoclax

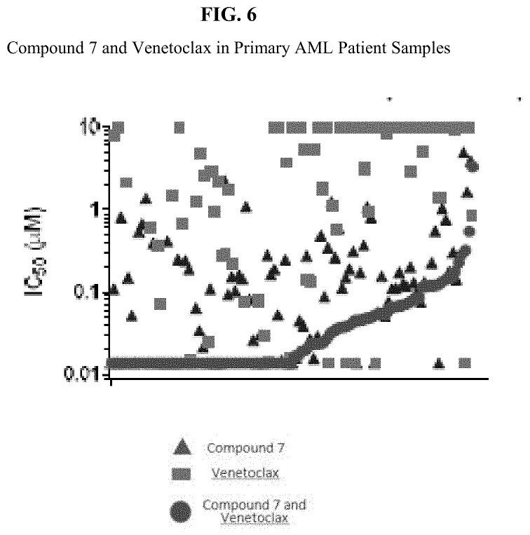

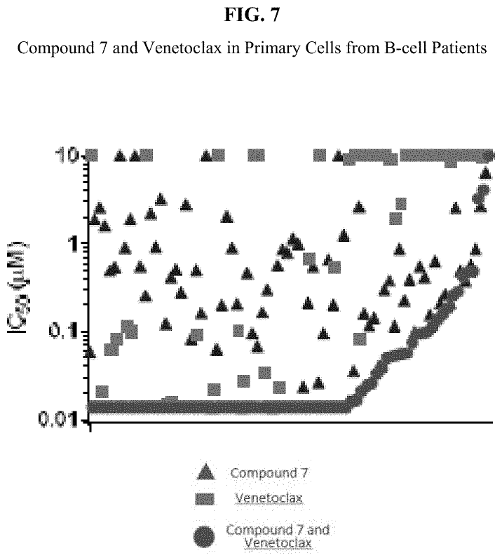

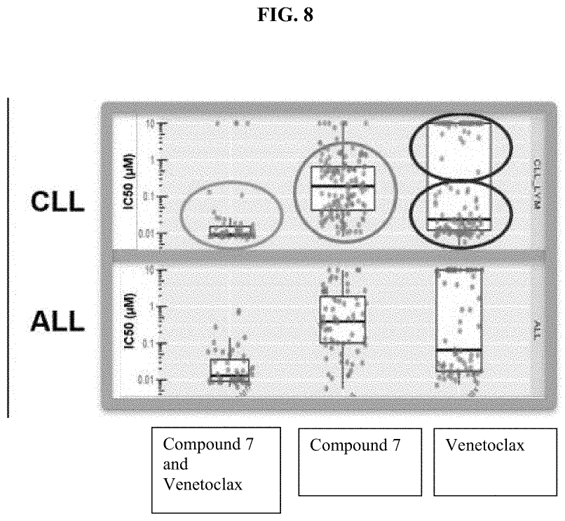

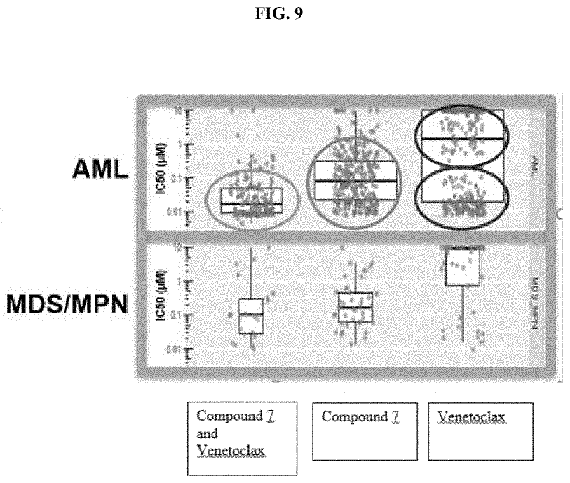

FIGS. 6-9 show that compound 7 and venetoclax synergistically kills primary cancer cell lines in multiple cancers, including AML, MDS, and B-cell cancers.

The publications discussed herein are provided solely for their disclosure prior to the filing date of the present application. Nothing herein is to be construed as an admission that the present invention is not entitled to antedate such publication by virtue of prior invention.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A pharmaceutical combination comprising a therapeutically effective amount of: 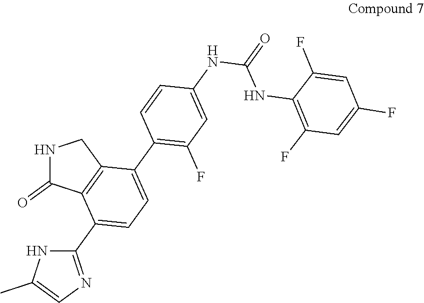 [SMILES: Cc1cnc(-c2ccc(-c3ccc(NC(=O)Nc4c(F)cc(F)cc4F)cc3F)c3c2C(=O)NC3)[nH]1]
or a pharmaceutically acceptable salt or solvate thereof, and at least one additional anticancer agent.

2. The pharmaceutical combination of claim 1, wherein the anticancer agent is a BCL-2 (B-cell lymphoma 2) protein inhibitor.

3. The pharmaceutical combination of claim 2, wherein the BCL-2 protein inhibitor is selected from one or more of the group consisting of venetoclax, navitoclax, and ABT-737.

4. The pharmaceutical combination of claim 3, wherein the combination is Compound 7 and venetoclax.

5. The pharmaceutical combination of claim 4, wherein the Compound 7 and venetoclax are both in an oral dosage form.

6. The pharmaceutical combination of claim 4, wherein the combination is a single pharmaceutical composition comprising both Compound 7 and venetoclax.

7. The pharmaceutical composition of claim 6, wherein the pharmaceutical composition is an oral dosage composition.

8. The pharmaceutical composition of claim 7, wherein the oral dosage composition is a tablet.

9. The pharmaceutical composition of claim 4, wherein Compound 7 and venetoclax are co-administered to a subject.

10. The pharmaceutical composition of claim 9, wherein Compound 7 and venetoclax are co-administered to a subject within the same day.

11. The pharmaceutical composition of claim 4, wherein the dosage amount of venetoclax is in the range of about 1 mg to about 150 mg.

12. The pharmaceutical composition of claim 4, wherein the dosage amount of Compound 7 is in the range of about 1 mg to about 300 mg.

13. The pharmaceutical composition of claim 7, wherein the dosage amount of venetoclax is in the range of about 1 mg to about 150 mg.

14. The pharmaceutical composition of claim 13, wherein the dosage amount of compound 7 is in the range of about 1 mg to about 300 mg.

15. A method of treating cancer in a subject, comprising administering to the subject in need thereof a therapeutically effective amount of Compound 7: 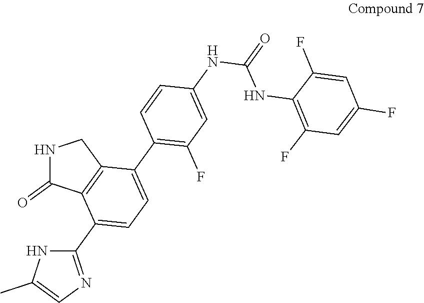 [SMILES: Cc1cnc(-c2ccc(-c3ccc(NC(=O)Nc4c(F)cc(F)cc4F)cc3F)c3c2C(=O)NC3)[nH]1]
or a pharmaceutically acceptable salt or solvate thereof, and at least one additional anticancer agent.

16. The method of claim 15, wherein the additional anticancer agent is venetoclax.

17. The method of claim 16, wherein the cancer is a hematological malignancy or B cell malignancy.

18. The method of claim 17, wherein the treated B cell malignancy is selected from one or more of the group consisting of mantle cell lymphoma (MCL), B-cell acute lymphoblastic leukemia (B-ALL), Burkitt's lymphoma, chronic lymphocytic leukemia (CLL), small lymphocytic lymphone (SLL), and diffuse large B-cell lymphoma (DLBCL).

19. The method of claim 16, wherein the cancer is a hematological malignancy.

20. The method of claim 19, wherein the hematological malignancy is leukemia.

21. The method of claim 20, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma.

22. The method of claim 21, wherein the leukemia is acute myeloid leukemia.

23. The method of claim 16, wherein the cancer is myelodysplastic syndromes (MDS) or myeloproliferative neoplasms (MPN).
